(19) United States
(12) Patent Application Publication
(10) Pub. No.: US 2022/0292572 A1
(43) Pub. Date: Sep. 15, 2022

(54) SYSTEM AND METHOD FOR CUSTOMIZING FORMULATIONS OF A CONSUMER CHEMICAL PRODUCT

(71) Applicant: BASF SE, Ludwigshafen (DE)

(72) Inventors: Jian Zhang, Shanghai (CN); Srikanth Vaduvur, Hong Kong (CN); Flavia Ferro, Hong Kong (CN); Kit Shan Cheung, Hong Kong (CN); Li Jun Chen, Shanghai (CN)

(21) Appl. No.: 17/637,502

(22) PCT Filed: Sep. 23, 2020
(86) PCT No.: PCT/EP2020/076482

§ 371 Date: Feb. 23, 2022

(30) Foreign Application Priority Data
Sep. 25, 2019 (CN) PCT/CN2019/107802

(51) Int. Cl. G06Q 30/06; G16C 20/20; G16C 20/90

(52) U.S. Cl. CPC G06Q 30/0631; G16C 20/20; G16C 20/90

(57) ABSTRACT

The present invention relates to a method for customizing formulations of a consumer chemical product, comprising the steps of: receiving a selection of navigation item, each of the navigation items directed at a strategy for customizing formulations of the consumer chemical product; receiving one or more inputs specifying characteristic information on the desired consumer chemical product and/or on the relevant personal features of the targeted consumers, and receiving one or more inputs for selecting the feedback on the existing consumer chemical products from the consumers or the markets, all the inputs being prepared under the selected navigation item; retrieving, from a first product database including product information of the existing consumer chemical products and preferences of consumers and market for the existing consumer chemical products, sub-items of the selected navigation item that meet the requirements as to the characteristic information; ranking the retrieved sub-items of the selected navigation item according to the feedback on the existing consumer chemical products from the consumers or the markets to provide a list of ranked sub-items of the selected navigation item; and recommending, from a second formulation database including a number of formulations different from those of the existing consumer chemical products, at least one candidate formulation according to one of top ranking sub-items in the list of ranked sub-items of the selected navigation item. The present invention further relates to a computer readable medium and a system comprising a server.

### TECHNICAL FIELD

The invention relates to a system and a method for customizing formulations of a consumer chemical product, particularly a care chemical product.

### BACKGROUND

Industries always try to meet consumers' needs, including catering for consumers' preference, in order to secure greater sales. In an era of digital revolution, consumer products may be acquired by digital pathways, thus rendering consumers' choices being more multiple. As a result, the consumers, while spending more and more money, diversify their needs, and even demand personified products in terms of performance and safety.

Particularly, in the consumer chemical industry, developing new products generally involves six stages: consumer research, data analysis, concept design, formulation research and design, launch preparation, and launch. All the stages of development are time-consuming and human-resource-intensive, and especially the analysis, and formulation research and design stages often involve large number of people with highly specialized skills and knowledge. For example, usually, a new care chemical product is launched after going through an expensive development cycle of generally 24-36 months more than 2 years, sometimes lagging behind the changing preference of customers.

In view of the rapid change in consumer choices and preferences, consumer chemical products are developed and put on the market at increasingly short development cycles. Thus, there exists a need to shorten the product development cycle of chemical products, while accommodating consumers' popular demands.

### SUMMARY

The invention encompasses a computer readable medium, a system, and a method for customizing formulations of a consumer chemical product. For the purpose of the present invention, consumer chemical products encompasses such products as care chemicals, pharmaceuticals, coatings, adhesives, sealants, inks, polishes, cleaners, and detergents.

In accordance with an aspect of the present invention, there is provided a method for customizing formulations of a consumer chemical product, comprising the steps of:
receiving a selection of navigation items, each of the navigation items directed at a strategy for customizing formulations of the consumer chemical product;
receiving one or more inputs specifying characteristic information on the desired consumer chemical product and/or on the relevant personal features of the targeted consumers, and receiving one or more inputs for selecting the feedback on the existing (i.e. existing on the market, the same hereinbelow, unless indicated otherwise) consumer chemical products from the consumers or the markets, all the inputs being prepared under the selected navigation item;
retrieving, from a first product database including product information of the existing consumer chemical products and preferences of consumers and markets for the existing consumer chemical products, sub-items of the selected navigation item that meet the requirements as to the characteristic information;
ranking the retrieved sub-items of the selected navigation item according to the selected feedback on the existing consumer chemical products from the consumers or the markets to provide a list of ranked sub-items of the selected navigation item; and
recommending, from a second formulation database including a number of formulations different from those of the existing consumer chemical products, at least one candidate formulation that corresponds to one of top ranking sub-items in the list of ranked sub-items of the selected navigation item.

In accordance with another aspect of the present invention, there is provided a system comprising a server, said server being in communication with one or more clients and one or more databases via a network, wherein said server receives and implements requests from the one or more clients for conducting the method of the invention. For the purposes of the present invention, the term “clients” includes but is not limited to a notebook, laptop, desktop or tablet computer device, mobile telephone, computing device technology, among any other arrangement that will be readily understood by the skilled person.

In accordance with a further aspect of the present invention, there is provided a computer readable medium having computer executable instructions which, when being executed upon a computer device, is adapted to perform the methods of the invention. Any suitable computer readable medium may be utilized including, but not limited to, random access memory (RAM), read only memory (ROM), electrically erasable programmable read only memory (EEPROM), flash memory or other memory technology, compact-disc read only memory (CD-ROM), digital versatile discs (DVD), holographic media or other optical disc storage, magnetic cassettes, magnetic tape, magnetic disk storage, and other magnetic storage devices.

These and other features, aspects and advantages of the present invention will become readily apparent to those skilled in the art and understood upon reading of this disclosure, and especially with reference to the following description, appended claims, and accompanying figures; but the invention is not limited to any particular disclosed embodiments.

### BRIEF DESCRIPTION OF THE DRAWINGS

For the purpose that the present invention can be understood in detail, a more particular description of the invention, briefly summarized above, may be made by reference to embodiments, some of which are illustrated in the appended drawings. It should be noted, however, that the appended drawings illustrate only typical embodiments of this invention and by no means limit the invention.

FIG. 1a illustrates a network environment in which a system in accordance with embodiments of the invention may be incorporated, and FIG. 1b illustrates a network environment in which a system in accordance with further embodiments of the invention may be incorporated.

FIG. 2 illustrates the flow chart of the general steps of a method for customizing formulations of a consumer chemical product in accordance with embodiments of the invention.

FIG. 3 illustrates the flow chart of a method for customizing formulations of a consumer chemical product in accordance with a further embodiment of the invention.

FIG. 4 illustrates a user interface for selecting a function module that is run by the system in accordance with embodiments of the invention.

FIG. 5 illustrates a user interface for selecting a sub-function module in the consumer insights module in accordance with an example of an embodiment of the invention.

FIGS. 6a-6c illustrate a user interface for market overview analysis in accordance with an example of an embodiment of the invention.

FIGS. 7a-7j illustrate user interfaces for customizing formulations of a consumer chemical product in accordance with embodiments of the invention.

FIGS. 8a-8b illustrate user interfaces for customizing formulations of a consumer chemical product in accordance with further embodiments of the invention.

FIG. 9 illustrates a user interface for customizing formulations of a consumer chemical product in accordance with still further embodiments of the invention.

FIGS. 10a-10b illustrate a user interface for formulation design in accordance with an example of a still further embodiment of the invention.

### DETAILED DESCRIPTION

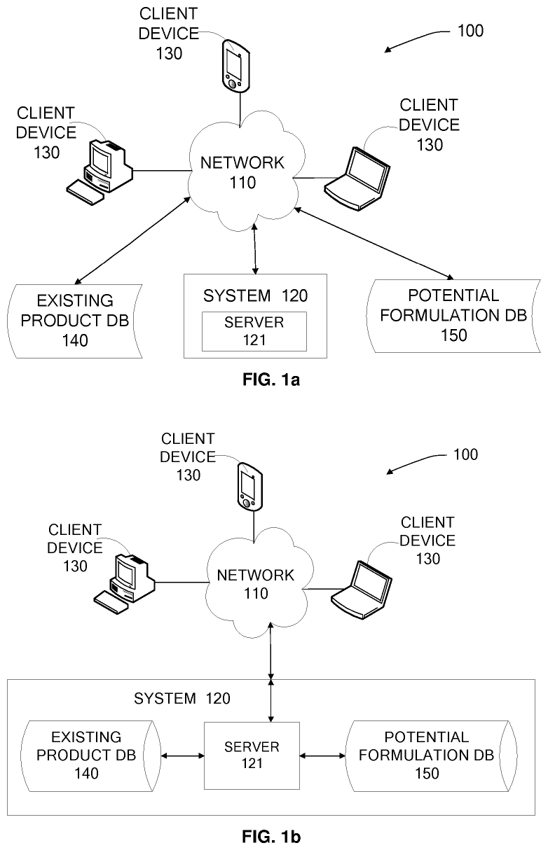

The present disclosure describes a method and a system for customizing formulations of a consumer chemical product. Several specific details of the invention are set forth in the following description and in FIGS. 1-10 to provide a thorough understanding of certain embodiments of the invention. One skilled in the art, however, will understand that the present invention may have additional embodiments, which falling within the scope of the claimed invention without departing from the spirit of the claimed invention.

In accordance with a first aspect of the present invention, there is provided a method for customizing formulations of a consumer chemical product, comprising the steps of:
receiving a selection of navigation items, each of the navigation items directed at a strategy for customizing formulations of the consumer chemical product;
receiving one or more inputs specifying characteristic information on the desired consumer chemical product and/or on the relevant personal features of the targeted consumers, and receiving one or more inputs for selecting the feedback from the consumers or the markets on the existing consumer chemical products, all the inputs being prepared under the selected navigation item;
retrieving, from a first product database including product information of the existing consumer chemical products and preferences of consumers and market for the existing consumer chemical products, sub-items of the selected navigation item that meet the requirements as to the characteristic information;
ranking the retrieved sub-items of the selected navigation item according to the selected feedback on the existing consumer chemical products from the consumers or the markets to provide a list of ranked sub-items of the selected navigation item; and
recommending, from a second formulation database including a number of formulations different from those of the existing consumer chemical products, at least one candidate formulation according to one of top ranking sub-items of the selected navigation item.

In more particular embodiments, if the selected navigation item is product or consumers' preference for products, the retrieved sub-items are the existing consumer chemical products. In that event, the step of recommending at least one candidate formulation according to one of the top ranking sub-items can comprise:
choosing, from the first product database, one of the top ranking products of the existing consumer chemical products that meet the requirements as to the characteristic information;
obtaining, from the first product database, key product information of the chosen top ranking product, and the key product information comprises one or more of at least one claimed product property, product form, product aspect, ingredients and application area; and
selecting, from the second formulation database, at least one candidate formulation that meet the requirements as to the key product information, for recommendation.

In a preferable embodiment, choosing one of the top ranking products of the existing consumer chemical products is choosing the first ranked product of the top ranking products, preferably automatically. Additionally or alternatively, one of the top ranking products may be chosen by the manufacturer and/or according to any other relevant criterion or criteria.

In a more particular embodiment, the step of recommending at least one candidate formulation according to one of the top ranking sub-items may further comprises, after choosing one of the top ranking products, providing at least one of the following information concerning the chosen top ranking product: ingredients feedback analysis, commenting consumers' locations, Top 10 comments, commenting consumers' skin types, commenting consumers' age, and consumers' preferences. In preferable embodiments, the feedback can be a 1 to 5-star rating system, which reflects the consumer satisfaction/preference score ranging between 1 star and 5 stars, with the higher feedback score representing the better consumer satisfaction. Furthermore, the commenting consumers' location can provide the live location like province where the consumer, who provides the comments, lives. The Top 10 comments can be the ten most popular keywords in consumers' comments. The comments may include, but not limited to, feeling, mildness, cleansing power, sensitivity, smell, and foam. The commenting consumers' skin type/age can provide the distribution of skin type/age of the consumers who provide comments. The consumers' preference, also known as consumers' insights, can provide information on clicks count at different time, for instance, in different years, or in different months. Moreover, the consumers' preference can also provide information on number of comments and comments rate at different locations, as well as the distribution of skin type/age of the consumers.

In a more particular embodiment, if the selected navigation item is product, the characteristic information may comprise one or more of brand, category, product form, product aspect, sunscreen levels, price, and claimed product properties. In some embodiments, the product form may include, but not limited to, Lotion, Oil, Liquid, Powder, Cream, Gel, Foam/Mousse, Aerosol and solid. In some embodiments, the product aspect may include, but not limited to, Ampoule, Capsule, Spray, Stick, Sheet, Wipe, Roll-on, Bar, Jar/Pot and Bottle. In some embodiments, the sunscreen levels may include, but not limited to, sunscreen level-SPF and sunscreen Level-PA. For instance, the sunscreen level-SPF may include Low (SPF≤10), Medium (10<SPF≤25), High (25<SPF≤50) and very High (SPF>50), and the sunscreen Level-PA may include PA+, PA++, PA+++ and PA++++.

In a further more particular embodiment, if the selected navigation item is consumers' preference for products, the characteristic information may comprise one or more of claimed product properties, category, skin type, gender, consumer area, age, and price.

In other particular embodiments, if the selected navigation item is consumers' preference for claimed product properties, the retrieved sub-items are the claimed product properties of the existing consumer chemical products. In that event, the step of recommending at least one candidate formulation according to one of the top ranking sub-items can comprises:
choosing, from the first product database, one of top ranking claimed product properties of the existing consumer chemical products that meet the requirements as to the characteristic information; and
selecting, from the second formulation database, at least one candidate formulation that has the chosen top rank claimed product property, for recommendation.

In a preferable embodiment, choosing one of the top ranking claimed product properties of the existing consumer chemical products is choosing the first ranked claimed product property of the top ranking claimed product properties, preferably automatically. Additionally or alternatively, one of the top ranking claimed product properties may be chosen by the manufacturer and/or according to any other appropriate criterion and criteria.

In an embodiment, the characteristic information comprises one or more of category, skin type, gender, consumer area, age, and price. The category may include, but not limited to, Color Cosmetics, Skincare, Hair Products, Soap & Bath Products, Oral Hygiene. The skin type may include, but not limited to, dry-rigid, dry-sensitive, oily-rigid, and oily-sensitive. The consumer area is where the target consumers are located/live. The sub-category of the category of skincare may include, but not limited to, Antiperspirant/Deo, Body Care, Cleansers/Make up remover, Essential Oil, Exfoliator/Blackhead Removal/Scrub, Eye Care, Face/Neck Care, Hand/Foot Care, Lip Care, Mask, Massage, Perfume/Parfum, Sets, Sun-After Sun and Sun-Sunscreen. The sub-category of the category of hair product may include, but not limited to, Hair Colorants, Hair Conditioner, Hair Styling/Relaxers, Hair treatment and Shampoo. The sub-category of the category of color cosmetics may include, but not limited to, Eye Brow, Eye Makeup, Face-Blush/Bronzer, Face-Foundations/Concealer, Face-Primer/illuminators, Lip Makeup and Nail color cosmetics. The sub category of the category of oral hygiene may include, but not limited to, Mouthwash and Toothpaste. The sub-category of the category of Soap & Bath products may include, but not limited to, shower products/soap.

In some embodiments, the at least one candidate formulation comprises category, application area, product form, claimed product properties, ingredients, specifications, and manufacturing process; and preferably the at least one candidate formulation further comprises at least one of sub-category, sub application area, product aspect, and additional performance.

In an embodiment, the method can further comprise highlighting ingredients that a designated supplier has, for example with a brand mark, in the at least one candidate formulation.

In some embodiments, the one or more inputs for selecting the feedback on the existing consumer chemical products from the consumers or the markets comprise the type of the feedback and optionally the period of the feedback. In a more particular embodiment, the type of the feedback is selected from clicks count, favorites count, and comments count. In a further particular embodiment, the type of the feedback is selected from clicks count, favorites count, comments count, and product updating time.

In some embodiments, the claimed product properties may include, but not limited to, Acne Scar Removal, After Sun Care, Alcohol Free, All-natural, Amino Acid-based, Anti Blue light, Anti Premature Aging, Youth preserve, Anti-Acne, Anti-aging, Anti-dandruff, Blackhead Removal, Body Shaping, Body Slimming, Boosting Elasticity, Botanical/Herbal, Breath Freshening, Brightening, Bust Beauty, Calming, Chinese Prescription, Color correcting, Concealer, Contouring and highlighting, Curl Enhancing, Dark Circle Removal, Dark Spot Removal, Deep Cleansing, Density Eyelash, Detoxify and purify, Even out skin tone, Exfoliating, Fast Absorbing, Firming, Fragrant, Gentle, Hair Brightening, Hair Care, Hair Color & Dye, Hair Discoloration Repair, Hair Loss Prevention, Hair Perm, Hair Putty, Hair Regrowth, Hair Removal, Hair Smooth, Hair Straightening, Hair Treatment, Hydrating, Irritation-free, Itching/Pruritus Treatment, Lightweight, Long-lasting, Meridian (Chinese medicine) Dredging, Moist, Moisturizing, Nourishing, Oil Control, Oil-Water Balance, Pain Relief, Preservative Free, Prickly Heat Treatment, Primer, Radiation Protection, Refreshing, Scar treatment, Silicone free, Skin Repair, Sleep Aids and Remedies, Smuggle-Proof, Soap Free, Soft texture, Sunscreen, Teeth Strengthening, Teeth Whitening, Tender, Ultraviolet-Proof, Under-eye bags Removal, Vitamin-based, Waterless, Waterproof, Weak Acids, Weak Alkaline, and Whitening. The lists of all the claimed product properties can be provided and selected in an alphabet order.

In some embodiments, the method further comprises the step of selecting the first product database from a plurality of databases. Furthermore, the method further comprises the step of updating the first product database from several data sources.

In preferable embodiments, the consumer chemical products can be care chemicals, preferably personal care chemicals and household chemicals, more preferably personal care chemicals.

In further embodiments, the method may further comprise the steps of:
receiving a request for trend searching, which comprises a selection of concepts of the desired consumer chemical product; and
providing the newest information concerning the selected concept in response to the request for trend searching.

In some embodiments, the request for trend searching further comprises one or more inputs for selecting the information on the chosen concept, preferably including at least one of source, segment, target demographic candidates, product focus and geographic regions, wherein the newest information meets the one or more inputs for selecting the information on the chosen concept.

In a further embodiment, the method may further comprise the steps of:
receiving a request for market overview analysis, which comprises one or more inputs for selecting the market overview; and
providing the market overview contents that meet the one or more inputs for selecting the market overview by surveying the first product database.

In a particular embodiment, the one or more inputs for selecting the market overview can comprise at least one of geographic regions, date, segment, and sub-segment; and the market overview contents can comprise at least one of retail market overview, segment retail values and growth rates, sub-segment retail values and growth rates, top 10 companies/brand retail value and market share, and company/brand performance.

In a further embodiment, the method may further comprise the steps of:
receiving a request for formulation design, which comprises a plurality of inputs for selecting formulation characteristics; and
recommending, from the second formulation database, at least one candidate formulation according to the selected formulation characteristics.

In a particular embodiment, the formulation characteristics comprise category, sub-category, product form, product aspect, claimed product properties, ingredient limitations, tags, and so on.

In a further embodiment, the method may further comprise the steps of:
receiving a selection of the ingredients in the at least one candidate formulation; and
providing characteristic information on the selected ingredient by surveying the first product database.

In a particular embodiment, the characteristic information on the selected ingredient comprises categories of the products containing the selected ingredient, functions of the selected ingredient, claimed ingredient properties, CAS (Chemical Abstracts Service) number, related trade names, registration area, consumers' preference (such as, clicks, favorites, comments) on the products containing the selected ingredient, number and ratio of the products containing the selected ingredient in each year, and number and ratio of the products containing the selected ingredient in each category during a selected year.

In a particular embodiment, the method may further comprise the steps of:
receiving one input specifying category of the products containing the selected ingredient and one input specifying the period of the feedback on the products containing the selected ingredient from the consumers or the markets;
receiving a selection of sorts of the feedback on the products containing the selected ingredient from the consumers or the markets;
retrieving, from the first product database, products containing the selected ingredient and under the specified category; and
ranking the retrieved products according to the selected sort of the feedback on the products containing the selected ingredient from the consumers or the markets during the specified period, to provide a list of ranked products.

In a further aspect of the present invention, it is provided a system comprising a server, said server in communication with one or more clients and one or more databases via a network; said server receives and implements requests from the one or more clients for conducting the method of the present invention.

The methods and systems disclosed herein may be implemented in any means for achieving various aspects, and may be executed in a form of a computer-readable medium embodying computer executable instructions that, when being executed upon a computer, cause the computer device to perform any of the steps disclosed herein.

The present invention is directed to an Internet-based platform that accumulates formulations (i.e., product specifications describing that the product is formulated from one or more components and working procedures to formulate at least a product) and the feedback on the existing consumer chemical products from the consumers or the markets (including, for example, retail value and growth rate, distribution profile of consumers, consumers' preference, behaviors, concerns, and so on). The Internet-based platform also accumulates a number of formulations different from those of the existing consumer chemical products, and these formulations are developed by specialty chemical suppliers, such as BASF. Accordingly, it functions as a repository of products, brands, ingredients and formulas for the manufacturer, such as product brand owners/original Equipment Manufacturers (OEMs)/original design manufacturers (ODMs), so that they can customize most appropriate formulations to meet the market requirements.

Further description of the invention may be made with reference to the attached figures.

FIGS. 1a-1b illustrate a network environment in which a system in accordance with embodiments of the invention may be employed. Of course, actual network and server environments can be arranged in a variety of configuration; however, the exemplary environment shown herein provides a framework for understanding the type of environment in which the present invention operates.

As shown in FIG. 1a, the network environment 100 may include a network 110, a system 120, clients 130, an existing product database 140, and a formulation database 150. The system 120 includes a server 121. In alternative embodiments, as shown in FIG. 1b, the system 120 includes the server 121, the existing product database 140 and the formulation database 150.

The network 110 is configured to facilitate communication between the clients 130 and the system 120, especially the server 121. The network 110 also facilitates communication between the server 121 and the existing product database 140, as well as communication between the server 121 and the formulation database 150. The network 110 may be a communication network, such as a wireless network, local area network, wired network, intranet, or the Internet. Server 121 provides data processing by way of server software such as database server system software. This data processing starts with the conversion of the data feeds to usable and harmonized data formats. Subsequently, the data will be mated (connect different data sets), washed (suppress unintended information), and finally refilled with additional or missing information. As such, the server 121 acts as a gatekeeper of data from a variety of data sources and provides that data to a variety of data consumers. Preferably, the server 121 is a cloud server; that is, the server software is host on a cloud platform. Either standardized Infrastructure as a Service (IaaS) or Platform as a Service (PaaS) may be leveraged to provide necessary application and database functionality.

The existing product database 140 may comprises detailed information of almost all of the existing products, including but not limited to information concerning chemical nomenclature, formulas, performance and sensory testing data, concept information, and user-related information such as users' behavior or users' comments. The existing product database 140 may also comprises market information, such as brand retail information, product properties claimed by the market players, brand information, and supply chain information. In some embodiments, the existing product database 140 may comprise a product database including product information on the existing products, and a feedback database including the feedback on the existing products from the consumers and the markets. Optionally or alternatively, the product database and the feedback database can be combined as one database, and the data stored therein can be linked by product information and/or personal features of consumers. For example, for care chemical products, data sources for the existing product database 140 may include formulation databases from care chemical companies, material databases from raw material suppliers, and commercial databases of feedback from consumers and markets; moreover, the existing product database 140 may also acquire data source from Chinese Food and Drug Administration (CFDA), Environmental Working Group (EWG), E-Commerce companies, and market intelligence agencies such as Mintel or EURO Monitor.

The formulation database 150 may comprises detailed information of a number of formulations different from those of the existing products, and these formulations are developed by specialty chemical suppliers. In some embodiments, the detailed information of these formulations may comprise category, application area, product form, claimed product properties, ingredients, specifications, and manufacturing process. In further embodiments, the detailed information of these formulations may further comprise at least one of sub-category, sub application area, product aspect, and additional performance. In some embodiments, the formulation database 150 may be enriched with new formulations designed by researchers and developers.

In some embodiments, the existing product database 140 and the formulation database 150 are local database; optionally or alternatively, the existing product database 140 and the formulation database 150 may be cloud database, i.e., the data therein are stored on the cloud.

In some embodiments, the clients 130 may employ a Web interface (e.g. Browser) or Mobile application (Android, iOS) to access the platform running on the server 121. The clients 130 may present the above-mentioned information in the databases 140 and 150.

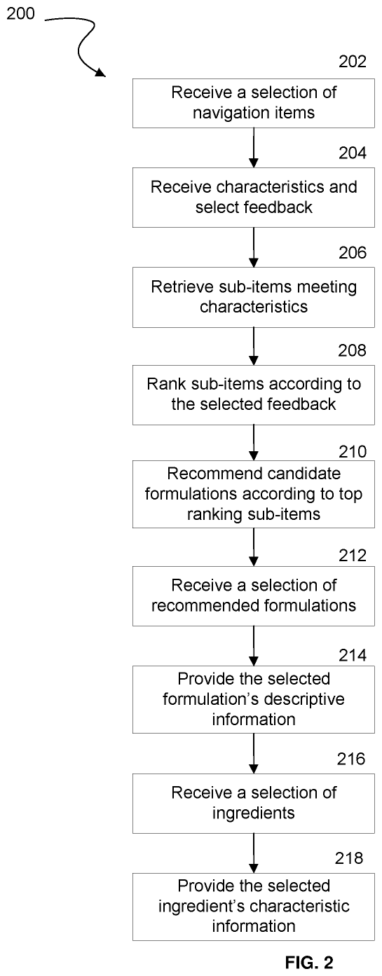

FIG. 2 illustrates the flow chart of the general steps of a method for customizing formulations of a consumer chemical product in accordance with embodiments of the invention. After receiving a selections of navigation items (step 202), a corresponding user interface is presented. The selected navigation item is directed at a strategy for customizing formulations of the consumer chemical product. In a particular embodiment, there are three navigation items, i.e. product, consumers' preference for products and consumers' preference for claimed product properties, respectively. Then, in this user interface, characteristic information on the desired consumer chemical product and/or the relevant personal features of the targeted consumers can be specified, and the feedback on the existing consumer chemical products from the consumers or the markets, which is also known as consumers' preference, such as clicks count, favorites count or comments count, can also be selected (step 204). It should be appreciate that the user interfaces of different customization strategies include different options for the characteristic information and the feedback. The characteristic information can be used to retrieve, from the existing product database 140, sub-items (such as, products or claimed product properties) that meet its requirements (step 206). The retrieved sub-items are ranked according to the selected feedback from the consumers or the markets to provide a list of ranked sub-items of the selected navigation item (step 208). Then, top ranking sub-items in the list of ranked sub-items are presented on this user interface. One of the top ranking sub-items may be chosen. In preferable embodiments, the first ranked sub-item is chosen automatically. Additionally or alternatively, one of the top ranking sub-items may be chosen by the manufacturer and/or according to any other appropriate criterion and criteria. Information about the chosen top ranking sub-item is used to select, from the formulation database 150, at least one candidate formulation for recommendation (step 210). In some embodiments, the server 121 may automatically select respective candidate formulations according to the information item(s), which is predetermined in the server 121, about the chosen top ranking sub-item.

In some embodiments, after the recommended formulations are obtained, one of the recommended formulations can be selected (step 212) to acquire its descriptive information (step 214), comprising category, application area, product form, claimed product properties, ingredients, specifications, manufacturing process, sub-category, sub application area, product aspect, additional performance and so on. Preferably, the ingredients that the designated supplier has may be highlighted, for example with the supplier's brand mark.

In a further embodiment, after the descriptive information on the selected formulation is acquired, one of the ingredients may be selected (step 216), for example by selecting its INCI (International Nomenclature Cosmetic Ingredient) name, and characteristic information on the selected ingredient is provided (step 218) by surveying the existing product database 140. The characteristic information on the selected ingredient may include, but not limited to, categories of the products containing the selected ingredient, functions of the selected ingredient, claimed ingredient properties, CAS number, related trade names, registration area, consumers' preference (such as, clicks, favorites, comments) on the products containing the selected ingredient, number and ratio of the products containing the selected ingredient in each year, number and ratio of the products containing the selected ingredient in each category during a selected year, and top 10 products containing the selected ingredient which is ranked according to the consumers' preferences or the markets' preferences.

In some embodiments, before step 204, a database as the existing product database 140 may be selected for getting the sub-items more appropriately. In order to acquire the newest information on the sub-items, the existing product database 140 can be updated, preferably in real time.

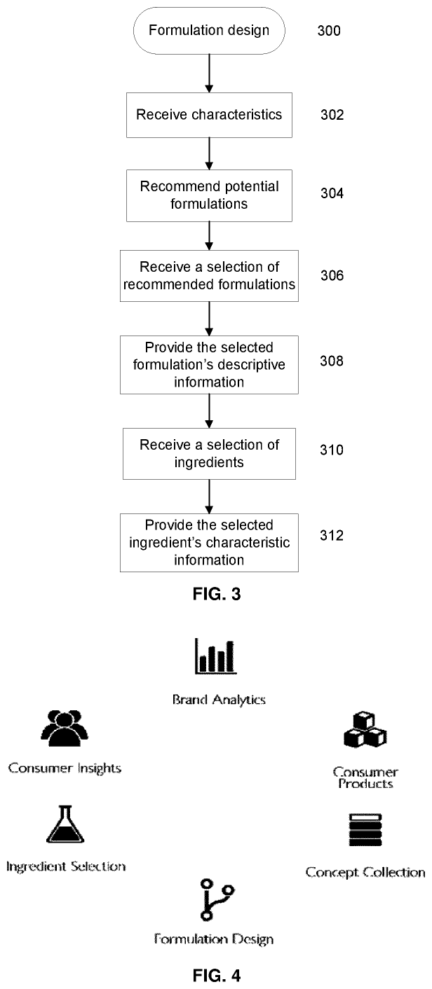

FIG. 3 illustrates the flow chart of another method for customizing formulations of a consumer chemical product, herein referred to as “formulation design”. If a navigation item of “formulation design” is selected (step 300), a “formulation design” user interface is presented. In this user interface, formulation characteristics, such as category, sub-category, product form, product aspect, claimed product properties, component limitations, tags, and so on, can be specified (step 302). The specified characteristics are used to select, from the formulation database 150, at least one candidate formulation for recommendation (step 304). It should be appreciated that, it is not necessary to specify all the formulation characteristics, and the more formulation characteristics are specified, the less the result formulations are, and vice versa. Similarly, after the recommended formulations are obtained, one of the recommended formulations may be selected (step 306) to acquire the descriptive information on the selected formulation (step 308). The descriptive information on the selected formulation may include, but not limited to, category, application area, product form, claimed product properties, ingredients, specifications, manufacturing process, sub-category, sub application area, product aspect and additional performance. Likewise, after the descriptive information on the selected formulation is acquired, one of the ingredients is selected (step 310), for example by selecting its INCI (International Nomenclature Cosmetic Ingredient) name, and characteristic information on the selected ingredient is provided (step 312) by surveying the existing product database 140.

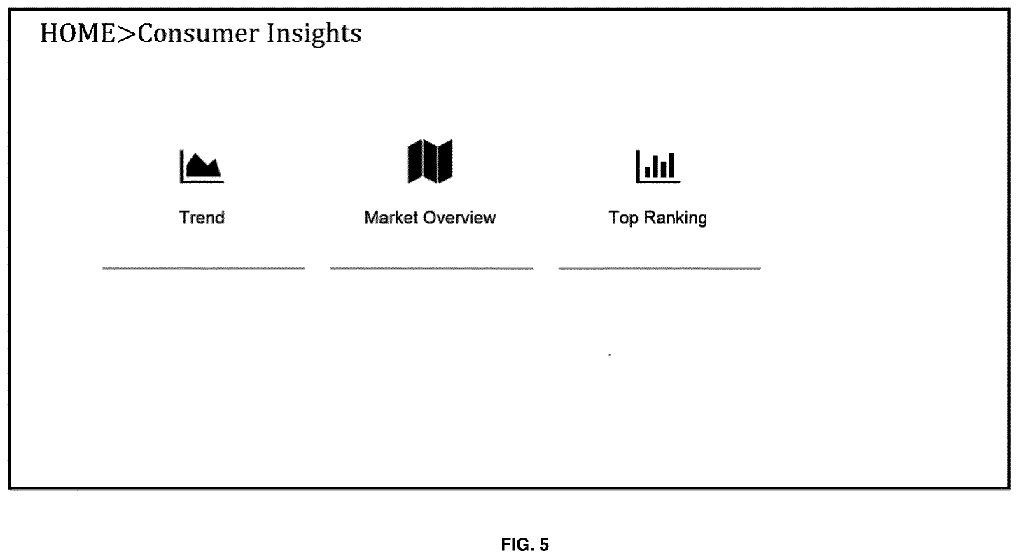

Now referring to FIGS. 4-10, the internet-based platform for a care chemical product example of embodiments of the present invention is described. FIG. 4 illustrates a user interface for selecting a function module that is run by the system in accordance with an example of embodiments of the invention. As shown in FIG. 4, the platform includes six modules: brand analytics, consumer products, concept collection, formulation design, ingredient selection, and consumer insights. If the consumer insights module is selected, a user interface for selecting a sub-function module in the consumer insights module is presented (for example, FIG. 5). In the user interface of FIG. 5, there are three sub-function modules: trend, market overview, and top ranking. If the manufacturers wish to learn the new product trend, they may enter the trend module, and thus a user interface for searching trend is presented (not shown). Firstly, an interesting concept of care chemical product (such as, protection, wellness, naturalness, Asia influence, professional beauty, beauty innovation, and personalization) listed on the interface may be chosen. There is a plurality of drop-down menus for selecting the information, including source, segment, target demographic candidates, product focus and geographic regions. In response to the request for trend searching, the newest information, such as press, presentation, paper etc., which meets the chosen concept and the selections of the plurality of drop-down menus, shall be presented below.

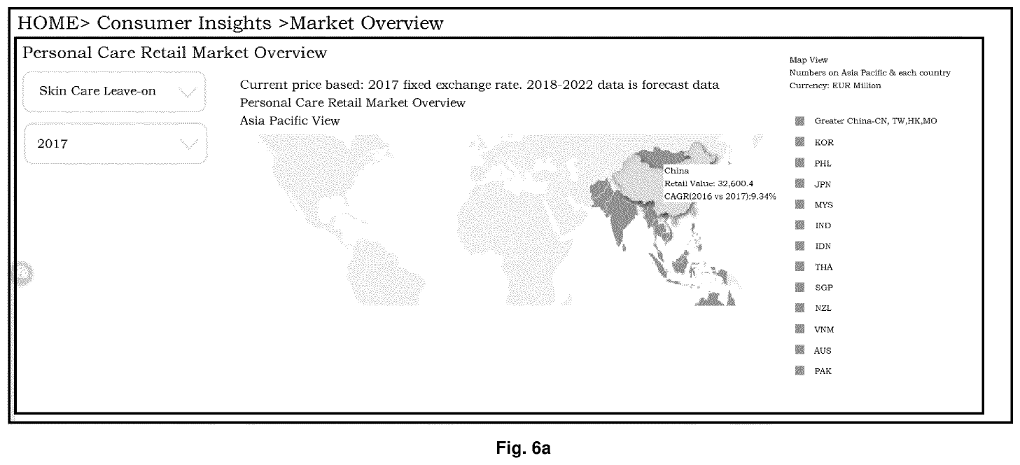

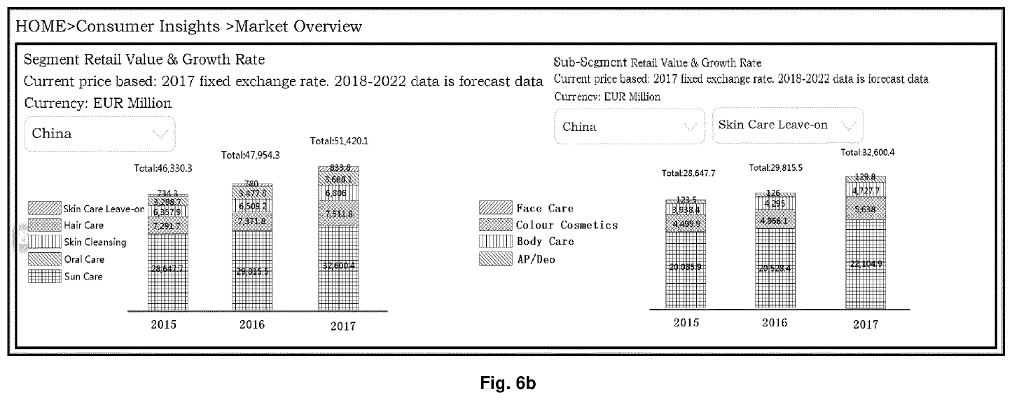

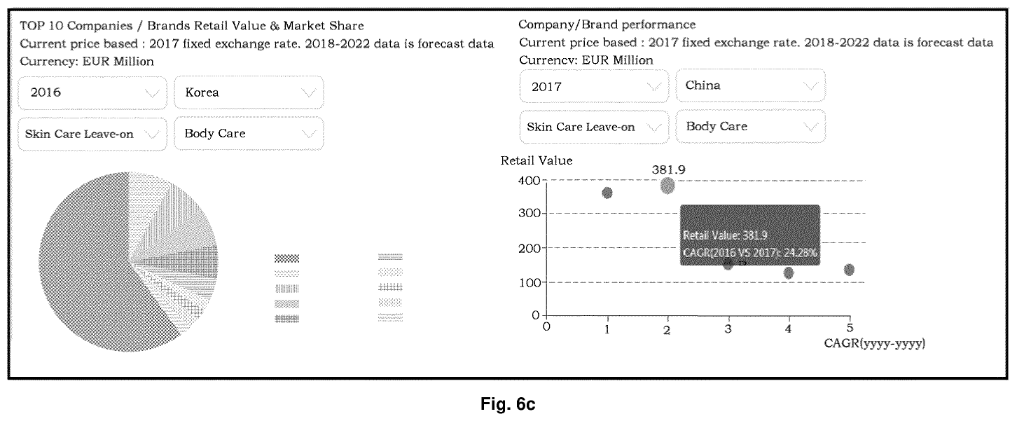

During product developing, it also needs to analyze the market overview. Accordingly, the market overview module in the user interface of FIG. 5 can be selected, and thus a user interface for market overview analysis is presented (for example, FIGS. 6a-6c). After specifying market overview requirements, market overview contents corresponding to the market overview requirements can be obtained from the existing product database 140.

FIG. 6a illustrates a world map divided by sales regions to show retail market overview of care chemical products selected in a segment drop-down menu during the selected year. FIG. 6b illustrates market overview contents of segment retail values & growth rates, and sub-segment retail values & growth rates. FIG. 6c illustrates market overview contents of top 10 companies/brand retail value & market share, and company/brand performance. In the left of FIG. 6b, a bar chart demonstrates retail values and growth rates of different segments (skin care leave-on, hair care, skin cleansing, oral care, and sun care) in different years at the geographic regions selected in a drop-down menu. In the right of FIG. 6b, a bar chart demonstrates retail values and growth rates of different sub-segments (face care, colour cosmetics, body care, AP/Deo) under the selected segment (skin care leave-on) in different years at the selected geographic region. In the left of FIG. 6c, a pie chart demonstrates retail value and market share of top 10 companies/brand in the selected segment and sub-segment during the selected year at the selected geographic region. In the right of FIG. 6c, a scatter plot demonstrates retail value and Compound Annual Growth Rate (CAGR) of companies/brands the selected segment and sub-segment in the selected year at the selected geographic region.

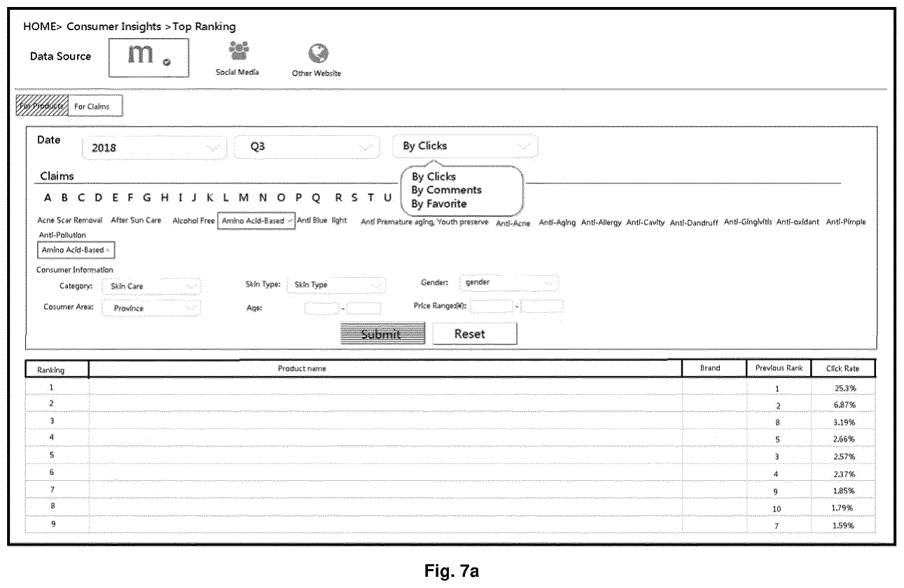

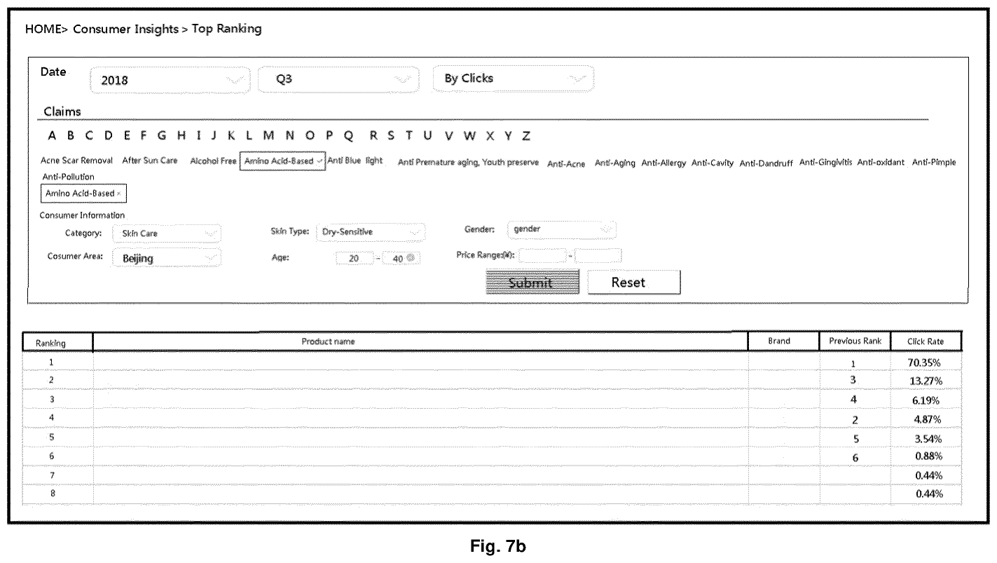

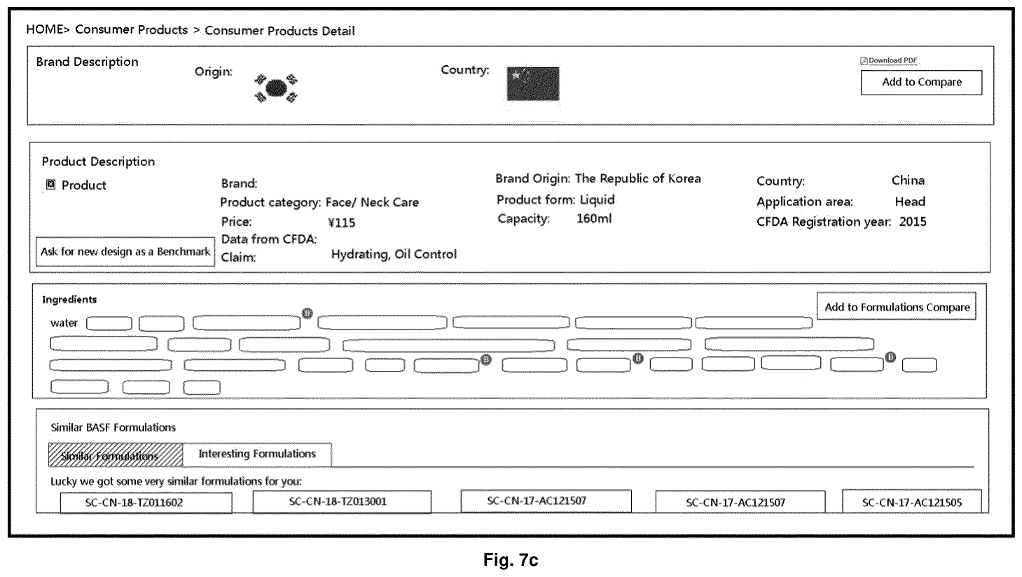

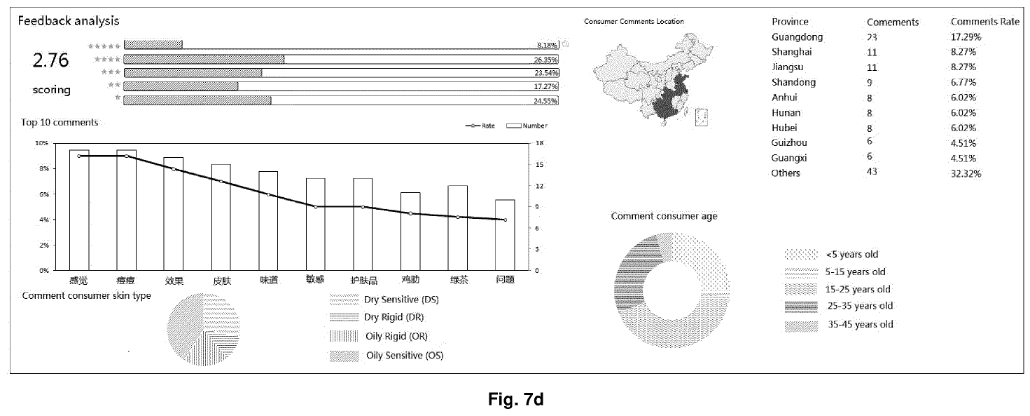

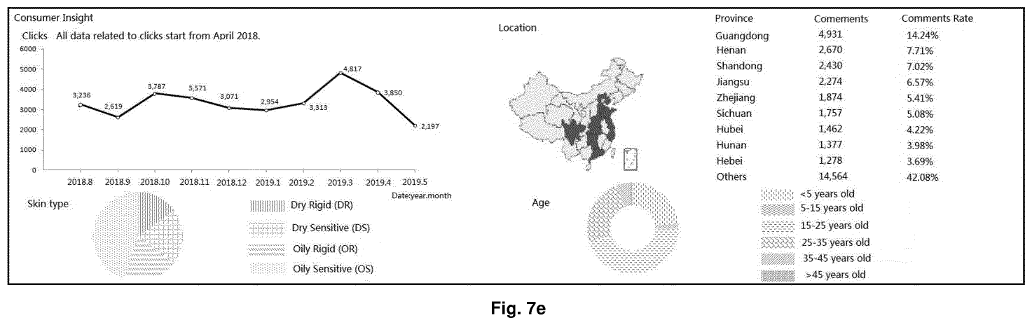

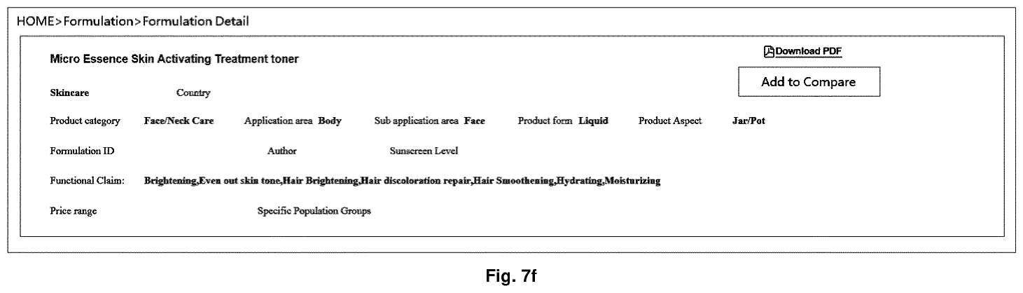

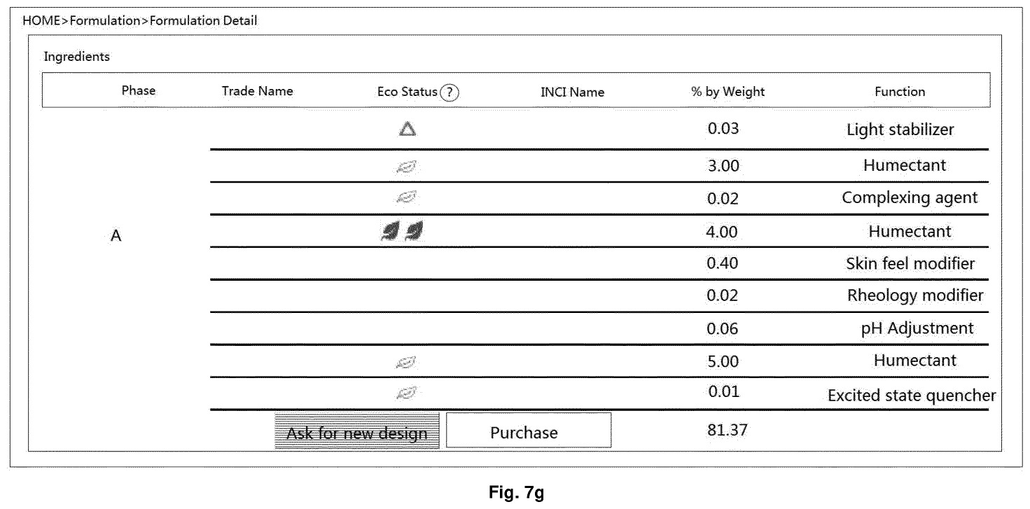

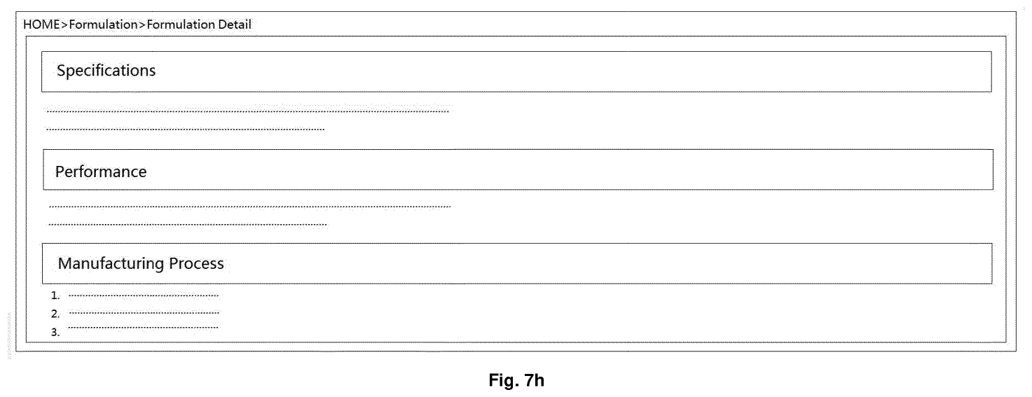

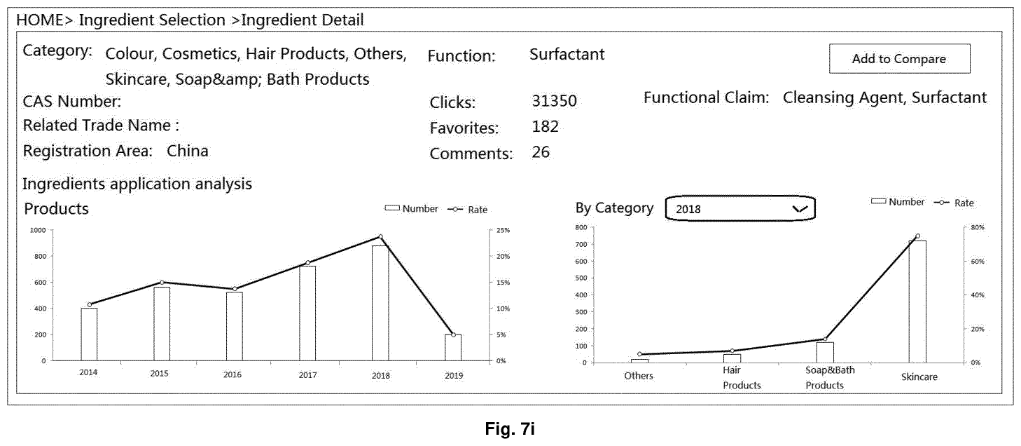

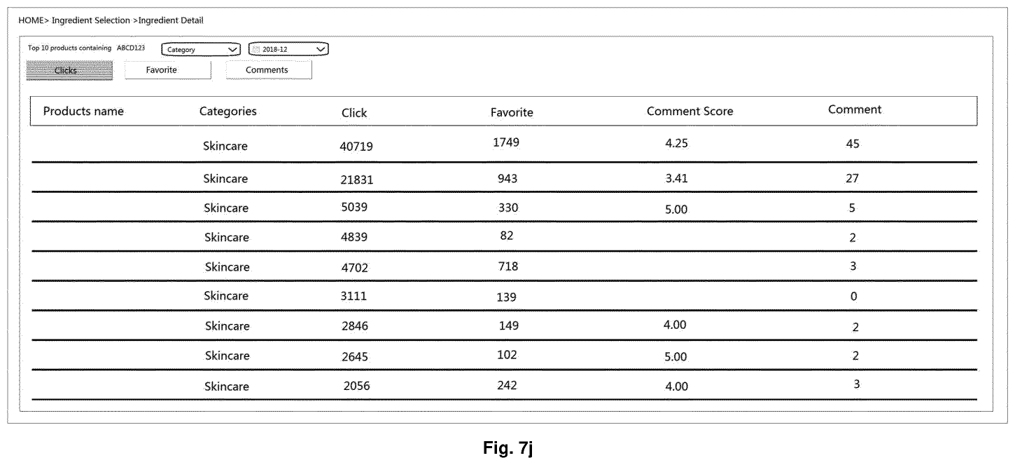

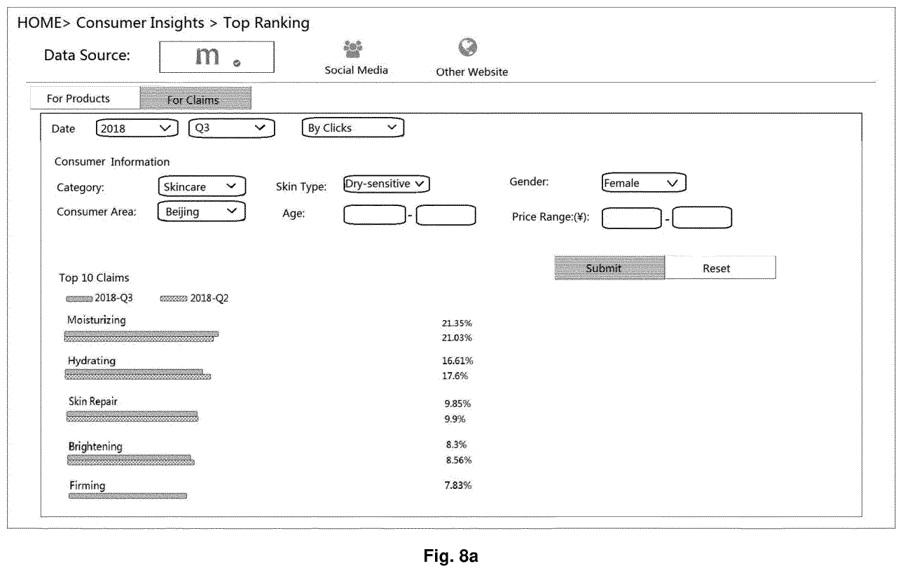

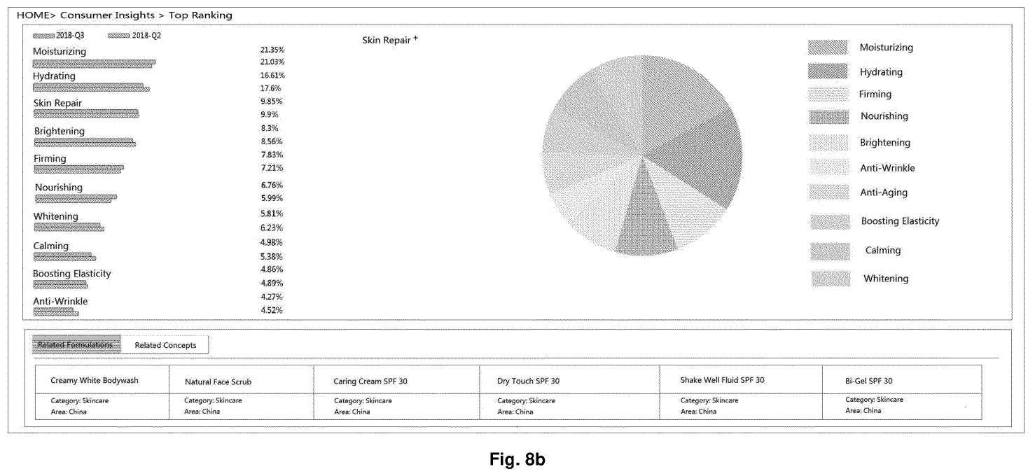

Referring back to FIG. 5, if the manufacturers wish to customize formulations according to top ranking, they may enter the top ranking module, and thus a user interface for analyzing top ranking is presented, such as shown in FIGS. 7a and 8a. On the top of this user interface, one of several data sources, such as social media and other website, can be selected as the source for obtaining the consumers' preference, also called “consumer's insights”. Next, one type of the consumers' preference, “for products” or “for claims”, may be selected as a ranking object, corresponding to the step 202 of FIG. 2.

Selection of a type of the consumers' preference causes a user interface demonstrating respective characteristic information options to be presented. For example, in FIG. 7a, under consumers' preference of “for products”, appropriate feedback on the existing consumer chemical products from the consumers or the markets, such as selecting the date and type of the feedback, may be selected to define the feedback that the manufacturer requires, and select appropriate characteristic information, including claims and consumer information, corresponding to the step 204 of FIG. 2. Each of the date and type of the feedback has an associated drop-down menu of selections. For example, selecting the drop-down box for the type of the feedback presents the selections for defining the ranking criterion, for example “by clicks”, “by comments” or “by favorites”. Similarly, drop-down boxes for year and quarter have associated selections for year and quarter of the feedback. On the other hand, the claimed product properties, also known as “claims”, are sorted by letter of an alphabet. Selecting a letter presents all the claims begin with this letter, and then one or more of the claims begin with the selected letter may be presented. For example, in FIG. 7a, the letter “A” is selected, and thus the claims begin with the letter “A”, such as Acne Scar Removal, After Sun Care, Alcohol Free, Amino Acid-Based, Anti Blue light, Anti Premature aging, Youth preserve, Anti-Acne, Anti-Aging, Anti-Allergy, Anti-Cavity, Anti Dandruff, Anti-Gingivitis, Anti-Pimple, Anti-Pollution, and Anti-Winkle, are presented. The selected claims would be presented and can be deleted by clicking the cross thereon. Moreover, in FIG. 7a, the consumer information includes category, skin type, gender, consumer area, age, and price. Drop-down boxes for category, skin type, gender, consumer area likewise have associated selections, respectively, whereas age and price can be set by inputting the range thereof.

After specifying the date and type of the feedback and the characteristic information, products meeting the requirements as to the specified characteristic information will be retrieved from the existing product database 140 (step 206), and be ranked according to the specified feedback (step 208). The top ranking products (for example, top ten products if existing) are presented. For example, in FIG. 7b, date is set to the third quarter, 2018, the type of the feedback is set to “by clicks”, the claimed product properties “Amino Acid-Based” is selected, the category is set to skin care, the skin type is set to dry-sensitive, the gender is set to female, the consumer area is set to Beijing, the age is set to 20-40, and no price is set. Accordingly, eight appropriate products are presented according to a ranking based on the clicks count. It should be appreciated that not all the characteristic information has to set. In the result ranking, presented are name, brand, previous rank and click rate of the products. By selecting one of the products presented, an additional detailed user interface is presented (e.g., user interface of FIGS. 7c-7e). In the detailed user interface, brand description (such as origin, country), product description (such as brand, brand origin, country, product category, product form, application area, price, capacity, CFDA registration year, data from CFDA, claimed product properties), and ingredients are presented.

Most importantly, candidate formulations, which are referred to herein as similar formulations, are presented in the detailed user interface, corresponding to step 210 of FIG. 2. These similar formulations are selected by comparing formulations in the formulation database 150 with key product information of the selected product. In preferable embodiments, the key product information comprises one or more of at least one claimed product property, product form, product aspect, and application area. As shown in FIG. 7c, the ingredients that BASF has are highlighted with BASF's brand mark, so as to facilitate selling BASF's products.

Moreover, in the detailed user interface, feedback analysis, commenting consumers' location, top 10 comments, commenting consumers' skin type, commenting consumers' age and consumers' preference are also presented (FIGS. 7d-7e). In FIG. 7d, a bar chart of percentage per star and a weighted scoring demonstrates the feedback analysis, a map demonstrates the number of comments and comments rate in each province, a plot with a horizontal axis of comments and a longitudinal axis of rate or number demonstrates the top 10 comments, a pie chart demonstrates rates of each skin type of the consumers who provide comments, and a doughnut chart demonstrates rates of each age range of the consumers who provide comments. The consumers' preference is presented in FIG. 7e, in which a plot with a horizontal axis of date and a longitudinal axis of clicks count demonstrates the clicks on the product at different time period, a map demonstrates the number of comments and comments rate on the product in each province, a pie chart demonstrates rates of each skin type of the consumers, and a doughnut chart demonstrates rates of each age range of the consumers.

One of the similar formulations in the detailed user interface may further be chosen, corresponding to step 212 or step 306. FIGS. 7f-7h present an example user interface that illustrates formulation's descriptive information, corresponding to step 214 or step 308. Here, the formulation's descriptive information comprises product category, sub-category, application area, sub application area, product form, product aspect, claims, ingredients (including trade name, eco status, INCI name, % by weight, function), specifications, additional performance, and manufacturing process.

In the ingredients column as shown in FIG. 7g, all the ingredients contained in the chosen similar formulation are present, and one of the ingredients may be selected by clicking its INCI name, corresponding to step 216 or step 310. In response to the click, an “ingredient selection” user interface (e.g., user interface of FIGS. 7i-7j) is presented (step 218 or step 312). The “ingredient selection” user interface includes characteristic information on the selected ingredient, which is obtained by surveying the existing product database 140. In FIG. 7i, categories of the products containing the selected ingredient, functions of the selected ingredient, claimed ingredient properties, CAS number, related trade names, registration area, consumers' preference (such as, clicks, favorites, comments) on the products containing the selected ingredient, and ingredients application analysis are present. The ingredients application analysis comprises a graph showing number and ratio of the products containing the selected ingredient in each year, and a graph showing number and ratio of the products containing the selected ingredient in each category during a selected year. In FIG. 7j, two drop-down boxes are presented for selecting the category of the products containing the selected ingredient and the period of the feedback on the products containing the selected ingredient from the consumers or the markets. Moreover, three indexing items (clicks, favorites, comments) representing the sorts of the feedback on the products containing the selected ingredient from the consumers or the markets are presented. After the category, the period and the sort of the feedback are selected, a list of top 10 products containing the selected ingredient and under the specified category are provided according to the selected sort of the feedback on the products containing the selected ingredient from the consumers or the markets during the specified period.

FIG. 8a presents a user interface of consumers' preference “for products”. Appropriate feedback on the existing consumer chemical products from the consumers or the markets, such as that specifying the date and type of the feedback, and appropriate characteristic information, including consumer information, may be specified to define the date and type of the feedback that the manufacturer requires, corresponding to the step 204 of FIG. 2. These are similar to that illustrated in FIG. 7a. After specifying the date and type of the feedback and the characteristic information, claims matching the specified characteristic information will be retrieved from the existing product database 140 (step 206), and be ranked according to the specified feedback (step 208). The top ranking claims (for example, top ten claims if existing) are presented. For example, in FIG. 8a, date is set to the third quarter, 2018, the type of the feedback is set to “by clicks”, the category is set to skin care, the skin type is set to dry-sensitive, the gender is set to female, the consumer area is set to Beijing, and no age constraint and price constraint is set. Accordingly, ten appropriate claims are presented according to a ranking based on the clicks count. In the result ranking, claim name and percentage under the selected quarter and the previous quarter are presented. For example, in FIG. 8b, top 10 claims are Moisturizing, Hydrating, Skin Repair, Brightening, Firming, Nourishing, Whitening, Calming, Boosting Elasticity, and Anti-Wrinkle. A pie chart on the right side demonstrates the percentage of each claim. In the meantime, similar formulations, which can also be referred to as candidate formulation, are presented in the user interface of FIG. 8b, corresponding to step 210 of FIG. 2. These similar formulations are selected by comparing formulations in the formulation database 150 with one of the top ranking claims, preferably the first ranked claim. One of the similar formulations may further be chosen, and formulation details may be presented similar to that shown in FIGS. 7f-7h. Moreover, one of the ingredients presented in the formulation details may be selected, and characteristic information on the selected ingredient may be presented similar to that shown in FIGS. 7i-7j.

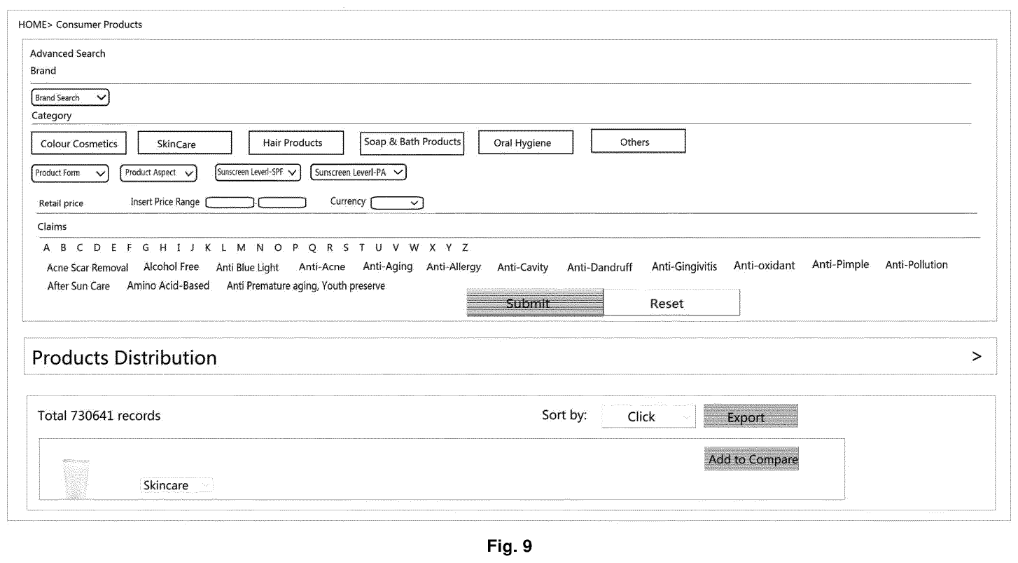

According to an embodiment of the invention, the consumer products module in the platform may be selected (in the user interface of FIG. 4), and a user interface for selecting existing product is presented (for example, FIG. 9). In the “consumer products” user interface, appropriate feedback on the existing consumer chemical products from the consumers or the markets (such as selecting the type of the feedback) and appropriate product characteristics, including brand, category, product form, product aspect, sunscreen levels (such as sunscreen level-SPF, sunscreen level-PA), retail price and claims, may be selected to define the manufacturer's needs, corresponding to the step 204 of FIG. 2. The type of the feedback has an associated drop-down menu of selections. For example, selecting the “sorted by” drop-down menu presents the selections from which the ranking criterion, for example “by clicks”, “by comments”, “by favorites” or “by updated time”, may be defined. Moreover, drop-down boxes for product form, product aspect, sunscreen levels have associated selections, respectively, whereas price can be set by inputting the range and currency thereof. Under category column, there are some category items, and the customer may select one or more of the category items. As mentioned above, the claims are sorted by a letter of an alphabet, and one or more claims may be selected. The selected claims and the selected category items would be presented and can be deleted by clicking the cross thereof.

After specifying the feedback and characteristic information, products meeting the requirements as to the specified characteristic information will be retrieved from the existing product database 140 (step 206), and be ranked according to the specified feedback (step 208). The top ranking products (for example, top ten products if existing) are presented. The description subsequent to presenting the top ranking products is similar to that mentioned above with reference to FIGS. 7c-7j.

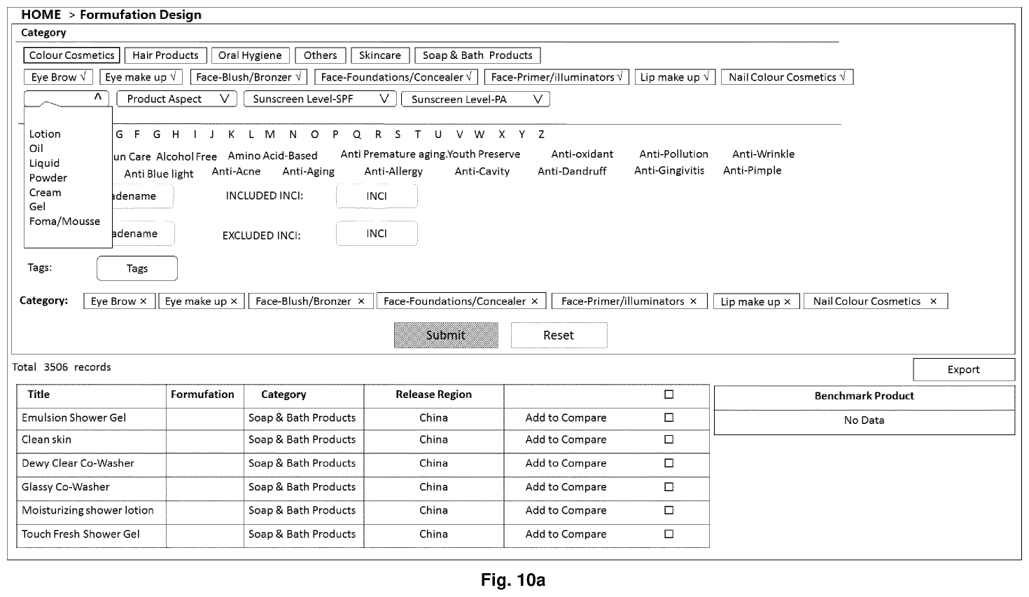

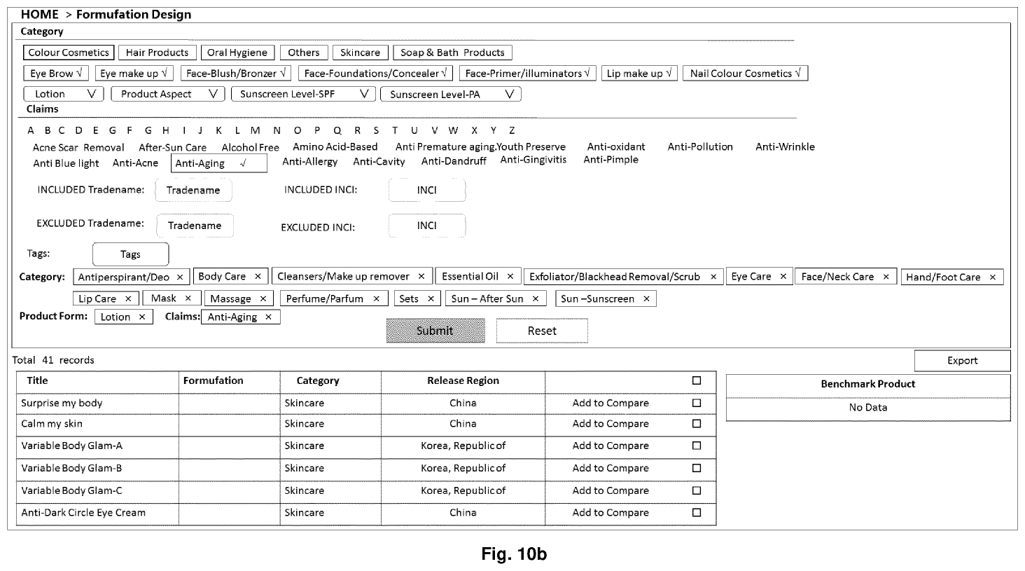

According to an alternative embodiment, the manufacturer may directly retrieve the formulation database 150 to obtain candidate formulations that the manufacturer needs. For this purpose, the formulation design module in the platform may be selected (in the user interface of FIG. 4), and a user interface for selecting candidate formulations is presented (for example, FIG. 10a). In the “formulation design” user interface, appropriate formulation characteristics, including category, sub-category, product form, product aspect, sunscreen levels (such as sunscreen level-SPF, sunscreen level-PA), claims, included tradename, excluded tradename, included INCI, excluded INCI and tags, may be specified to define the manufacturer's needs. Under category column, there are some category items. Once a category item is selected, several sub-category items under the selected category are presented and are selected automatically. The subcategories selected can be deleted by clicking the cross on the subcategories presented below. As mentioned above, drop-down boxes for product form, product aspect, sunscreen levels have associated selections, respectively, whereas the claims are sorted by a letter of an alphabet. Moreover, drop-down boxes for included tradename, excluded tradename, included INCI, excluded INCI and tags also have associated selections, respectively.

After specifying formulation characteristics, candidate formulations meeting the specified formulation characteristics will be retrieved from the formulation database 150 and presented below. For example, in FIG. 10b, the category of skincare is selected, and the all the sub-category items (e.g., Antiperspirant/Deo, Body Care, Cleansers/Make up remover, Essential Oil, Exfoliator/Blackhead Removal/Scrub, Eye Care, Face/Neck Care, Hand/Foot care, Lip Care, Mask, Massage, Perfume/Parfum, Sets, Sun-After Sun, and Sun-Sunscreen) under skincare are selected. Furthermore, the claim of Anti-Aging is selected, and the product form is set to Lotion. Accordingly, totally 41 candidate formulations are presented below. The description subsequent to presenting the candidate formulations is similar to that mentioned above with reference to FIGS. 7f-7j.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of and is not intended to convey an indication of a preferred or ideal embodiment. “Such as” is not used in a restrictive sense, but for explanatory purposes.

It should be noted that the above-mentioned examples illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfill the functions of several units recited in the claims. Any reference signs in the claims shall not be construed so as to limit their scope.

1. A method for customizing formulations of a consumer chemical product, comprising:
receiving a selection of navigation items, each of the navigation items directed at a strategy for customizing formulations of the consumer chemical product;
receiving one or more inputs specifying characteristic information on the desired consumer chemical product and/or on relevant personal features of targeted consumers, and receiving one or more inputs for selecting a feedback on existing consumer chemical products from consumers or markets, all the inputs being prepared under the selected navigation item;
retrieving, from a first product database including product information of the existing consumer chemical products and preferences of the consumers and the market for the existing consumer chemical products, sub-items of the selected navigation item that meet the requirements as to the characteristic information;
ranking the retrieved sub-items of the selected navigation item according to the selected feedback on the existing consumer chemical products from the consumers or the markets to provide a list of ranked sub-items of the selected navigation item; and
recommending, from a second formulation database including a number of formulations different from those of the existing consumer chemical products, at least one candidate formulation according to one of the top ranking sub-items in the list of ranked sub-items of the selected navigation item.

2. The method of claim 1, wherein if the selected navigation item is product or consumers' preference for products, the retrieved sub-items are the existing consumer chemical products.

3. The method of claim 2, wherein the step of recommending at least one candidate formulation according to one of the top ranking sub-items comprises:
choosing, from the first product database, one of top ranking products of the existing consumer chemical products that meet the requirements as to the characteristic information;
obtaining, from the first product database, key product information of the chosen top ranking product, wherein the key product information comprises one or more of at least one claimed product property, product form, product aspect, ingredients and application area; and
selecting, from the second formulation database, at least one candidate formulation that meet the requirements as to the key product information, for recommendation.

4. The method of claim 3, wherein choosing one of the top ranking products of the existing consumer chemical products is choosing the first ranked product of the top ranking products.

5. The method of claim 3, the step of recommending at least one candidate formulation according to one of the top ranking sub-items further comprises, after choosing one of the top ranking products, providing at least one of the following information concerning the chosen top ranking product: Ingredients, feedback analysis, commenting consumers' location, Top 10 comments, commenting consumers' skin type, commenting consumers' age, and consumers' preference.

6. The method of claim 1, wherein if the selected navigation item is product, the characteristic information comprises one or more of brand, category, product form, product aspect, sunscreen levels, price, and claimed product properties.

7. The method of claim 1, wherein if the selected navigation item is consumers' preference for products, the characteristic information comprises one or more of claimed product properties, category, skin type, gender, consumer area, age, and price.

8. The method of claim 1, wherein if the selected navigation item is consumers' preference for claimed product properties, the retrieved sub-items are the claimed product properties of the existing consumer chemical products.

9. The method of claim 8, wherein the step of recommending at least one candidate formulation according to one of the top ranking sub-items comprises:
choosing, from the first product database, one of top ranking claimed product properties of the existing consumer chemical products that meet the requirements as to the characteristic information; and
selecting, from the second formulation database, at least one candidate formulation that has the chosen top rank claimed product property, for recommendation.

10. The method of claim 9, wherein choosing one of the top ranking claimed product properties of the existing consumer chemical products is choosing the first ranked claimed product property of the top ranking claimed product properties.

11. The method of claim 9, wherein the characteristic information comprises one or more of category, skin type, gender, consumer area, age, and price.

12. The method of any of claim 1, wherein the at least one candidate formulation comprises category, application area, product form, claimed product properties, ingredients, specifications, and manufacturing process; and optionally the at least one candidate formulation further comprises at least one of sub-category, sub application area, product aspect, and additional performance.

13. The method of any of claim 1, wherein the one or more inputs for selecting the feedback on the existing consumer chemical products from the consumers or the markets comprise the type of the feedback and optionally the period of the feedback.

14. The method of claim 13, wherein the type of the feedback is selected from clicks count, favorites count, comments count, and optionally product updating time.

15. The method of any of claim 1, further comprising selecting the first product database from a plurality of databases.

16. The method of any of claim 1, further comprising updating the first product database from several data sources.

17. The method of any of claim 1, wherein the consumer chemical products are care chemicals.

18. A system comprising a server, said server in communication with one or more clients and one or more databases via a network; wherein said server receives and implements requests from the one or more clients for conducting the method of claim 1.

19. A computer readable medium having computer executable instructions which, when being executed upon a computer device, is adapted to perform the method according to claim 1.

20. The method of claim 17 wherein the care chemicals are personal care chemicals or household chemicals.
